(19) United States
(12) Patent Application Publication
(10) Pub. No.: US 2002/0147235 A1
(43) Pub. Date: Oct. 10, 2002

(54) Stable aqueous antimicrobial suspension

(75) Inventors: Paul E. Carlson, Pittsburgh, PA (US); H. Edwin Nehus, Pittsburgh, PA (US); George Bell, Pittsburgh, PA (US)

Correspondence Address: William L. Krayer, 1771 Helen Drive, Pittsburgh, PA, 15216, US

(73) Assignee: Verichem, Inc.

(21) Appl. No.: 10/052,115

(22) Filed: Jan. 17, 2002

Related U.S. Application Data
(60) Provisional application No. 60/264,611, filed on Jan. 27, 2001.

(51) Int. Cl. A61K 31/275

(52) U.S. Cl. 514/528

(57) ABSTRACT

Suspensions of haloacetamides are made and stabilized with a xanthan gum containing no more than 1.2% by weight acetic acid or acetate groups, together with a buffer comprising sodium acetate and acetic acid. The process and composition is especially effective for DBNPA:2,2-dibromo-3-nitrilopropionamide.

### RELATED APPLICATION

This application claims the benefit of our Provisional patent application of the same title filed Jan. 27, 2001.

### TECHNICAL FIELD

This invention relates to antimicrobial compositions, and particularly to aqueous-based compositions which are stable and effective over long periods.

1. Background of the Invention

Haloacetamides are used extensively as antimicrobial agents in various industrial applications, such as water treatment and preservation. The active ingredient (the haloacetamide) is a solid, which is difficult to feed in industrial applications and poses problems in material handling. Because of the problems in handling solids, liquid concentrates have been developed. Such liquid concentrates are convenient for their ability to be diluted, and their relative ease of application.

While it is desirable to make and use haloacetamides in liquid form, it has been difficult to formulate a stable aqueous formulation. Haloacetamides decompose rapidly by hydrolysis or photolysis. Moreover, most suspending agents tend to break down under acidic conditions. Currently used commercial formulations utilize a mixture of organic solvents and water, or, because of the proclivity of the haloacetamide to hydrolyze, sometimes the solvent without water, to carry the haloacetamides. Users have raised concerns about the organic solvents because of their toxicity to man by occupational exposure and to the environment.

Xanthate gum has been proposed for use as a thixotropic suspending agent for suspensions of 2,2-dibromo-3-nitrilopropionamide (DBNPA) by Gartner in U.S. Pat. No. 5,627,135. However, Miskiel and Solanki, in U.S. Pat. No. 6,083,890, have shown that acidic cleaning compositions containing Xanthan gum and a preservative (5-bromo-5-nitro- 1,3-dioxane) rapidly lose viscosity, while a low-acetate Xanthan gum maintained the viscosity stability or even increased it. See Table 1 of U.S. Pat. No. 6,083,890. The natural Xanthan gum, containing at least 5% acetic acid groups, typically 5.6% by weight, itself degrades in an acidic environment. As reviewed by Miskiel and Solanki column 3, lines 33-47, “Although xanthan gum is well known as a rheology modifier in cleansers, characteristically the viscosity decreases undesirably over time at low pH, within about seven days after making the compositions. The extent to which the viscosity decreases is dependent on a number of factors, such as the pH and ionic strength of the cleaner and the pH levels, and the temperature of the acidic cleaner composition at which it is stored. In compositions stored at ambient temperature, xanthan gum loses a significant proportion, perhaps greater than about 20% or more, of its viscosifying functionality within an acidic composition in about seven days at a pH of about 2.2 or less. This may eventually lead to product performance disappointment and failure unless an increased concentration of xanthan gum is initially used to compensate for the decrease in viscosity.”

The difficulty of creating a stable suspension of a haloacetamide with Xanthan gum is compounded by the fact, as mentioned above, that the haloacetamides tend to hydrolyze in water and especially so at higher pH's. Thus the desirability of a low pH to preserve the haloacetamide conflicts with the adverse effects of a low pH on a suspending agent such as natural Xanthan gum. Nevertheless, Gartner, in U.S. Pat. No. 5,627,135, recommends reducing the pH of the water to below 7 before adding the natural Xanthan and says that “the pH of the formulation will usually equiibrate to about 1 to about 4 and no further acidification is needed.” Col 5 lines 34-51. His Table 1, however, contains no examples using xanthan gum alone as the suspending agent.

An acid stable liquid formulation of a haloacetamide is needed in the industry. The need is especially critical for a stable formulation of 2,2 dibromo 3-nitrilopropionamide (“DBNPA”).

2. Summary of the Invention

This invention includes a formulation of an aqueous suspension or dispersion of haloacetamide that only uses water as the solvent and is stable when stored. The invention uses a unique agent capable of suspending haloacetamides over a broad range of concentration, inhibiting hydrolysis. The haloacetamides are preferably suspended in concentrations from 5% to 60% by weight, although higher concentrations can be used where high viscosities can be tolerated.

To suspend the formulations, an acetate-free Xanthan gum is used in a concentration ranging from 0.1% to 5%, anchoring the pH between 1 and 5 with a buffer comprising sodium acetate and acetic acid in a weight ratio of 1.5:1 to 2.5:1, in an amount effective to maintain the pH between 1 and 5 for a desired period of stability.

The invention provides:

a. Storage Stability equivalent to other commercially available solutions.

b. Equivalent microbiological efficacy to other commercially available formulations over the use of the formulation.

c. Reduces toxicity of the formulation when composed to other commercial formulations

d. Eliminate the use of undesirable solvents.

By an acetate-free xanthan gum, we mean a xanthan gum which contains in its molecular structure no more than 1.5% acetic acid and/or acetate groups. Such a material may be made by deacetalating natural xanthan gum as disclosed in any of U.S. Pat. Nos. 3,096,293, 4,214,912, 4,369,125, 4,873,323 or by any other suitable method which does not destroy the viscosifying ability of the xanthan, i.e. which is substantially undegraded as described by Miskiel and Solanki U.S. Pat. No. 6,083,890, column 6, lines 29-44. Preferably the acetate-free xanthan gum will have no more than 1.2% acetic acid, more preferably no more than 0.6%, and most preferably 0% (as a practical matter, no more than 0.1%) by weight acetate or acetic acid groups. A zero percent content may be found in xanthan gums made by “certain genetically modified Xanthomonas species which lack the necessary acetyltransferase genes required to transfer these moieties as substitutents to the side chains of the xanthan gum molecule” (column 6, lines 64-67, Miskiel and Solanki U.S. Pat. No. 6,083,890). Both the Miskiel and Sloanki U.S. Pat. No. 6,083,890 and Gartner U.S. Pat. No. 5,627,135 are incorporated herein in their entireties.

Thus our invention includes a stable liquid formulation of a haloacetamide comprising, in water, at least 5% by weight haloacetamide (preferably 5% to 60%, more preferably 10% to 45% and most preferably 15% to 25% by weight), 0.1% to 5% by weight (preferably 0.5% to 4%) of an acetate-free Xanthan gum suspending agent, and acetic acid, sodium acetate or a mixture thereof as a buffering agent effective to maintain the suspension at a pH between 1 and 5, preferably between 3.8 and 4.2. Typically, an effective amount of buffering agent will comprise 1-2% sodium acetate and 0.5-1% acetic acid, preferably in a weight ratio of 1.5:1 to 2.5:1. Our invention includes a method of making the suspension, comprising forming an aqueous solution of 0.1% to 5% by weight of an acetate-free xanthan gum, adding the buffer, and then adding the haloacetamide in the proportions desired to make a composition as described above. The buffer as added not merely to reduce the initial pH (cf Gartner U.S. Pat. No. 5,627,135 col 5 lines 34-50) but to maintain it over a period of time to inhibit hydrolysis of the DBNPA.

Our invention is applicable to any of the halogenated amides recited in Burk et al U.S. Pat. No. 4,163,798, which is incorporated herein by reference in its entirety. In particular, the halogenated amides useful in our invention are alpha-haloamides; that is, compounds which contain an amide functionality [ie a moiety of the formula —C(O)—N<] and which have at least one halogen atom on a carbon atom located adjacent to (that is, in the alpha position relative to) the carbonyl group [—C(O)—] of such amide functionality. Preferably, they are halogenated nitrilopropionamides. Examples of the preferred group are 2,2 dibromo 3-nitrilopropionamide (“DBNPA”), 2-bromo-2-cyano-N,N-dimethylacetamide, 2-bromo 3-nitrilipropionamide, 2-bromo 2,3-dinitrilipropionamide, N,N-dimethyl-2,2-dibromo-3-nitrilipropionamide, and N-(n-propyl)-2-iodo-2bromo-3-nitrilopropionamide A most preferred haloacetamide is 2,2 dibromo 3-nitrilipropionamide (“DBNPA”). A preferred buffering agent comprises sodium acetate and acetic acid, preferably in a molar ratio of 1.5:1 to 2.5:1, and more preferably about 2:1.

Suspensions and/or dispersions of the above described formulations are stable and effective over long periods of time, are conveniently prepared and dispensed for use, and are more acceptable environmentally and with respect to toxicity than comparable conventional antimicrobial compositions.

### BRIEF DESCRIPTION OF THE DRAWINGS

FIG. 1 shows graphically the known rate of hydrolysis of DBNPA at 25° C.

### DETAILED DESCRIPTION OF THE INVENTION

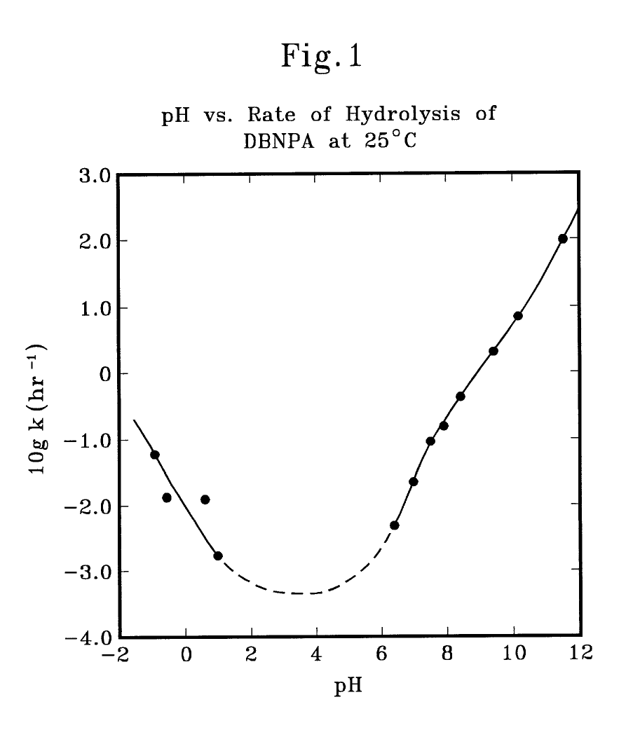

FIG. 1 is a plot of the known hydrolysis in water of DBNPA. It will be seen that the lowest rate of hydrolysis is at slightly less than pH 4.

Table 1 below shows the results of several experiments testing the physical and chemical stability of our compositions. For these tests, suspensions were made, according to the procedure described above, of haloacetamide using acetate-free xanthan as the suspending agent and various additives intended as buffering agents. The procedure was designed to comply with the U.S. EPA Product Properties Guidelines, 830.6317; see part (c), accelerated at 50° C. All samples utilized 20% DBNPA except one which employed 20% 2-bromo-2-cyano-N,N-dimethylacetamide as the haloacetamide. Physical stability was determined visually; chemical stability was determined by pH and titration.

TABLE 1

| | | | Days | |
|---|---|---|---|---|
| | | | stable, | Days stable, |
| AFX¹, wt % | Buffer | Buf. Conc. | physical | chemical |
| 0.4 | OX ACID² | 0.1 M | 1 | 9 |
| 0.6 | AcOH, NaAc³ | 1%, 0.1% | 22 | 29 |
| 0.6 | AcOH, NaAc | 0.5%, 2% | 25 | 25 |
| 0.5 | AcOH, NaAc | 0.5%, 1% | 11 | 17 |
| 0.6 | AcOH, NaAc⁴ | 0.1%, 2% | 14 | 35 |
| 0.6 | AcOH, NaAc⁵ | .508%, 1.01% | 32 | 32 |
| 0.6 | AcOH | 0.2 M | 27 | 27 |
| 0.6 | AcOH | 0.1 M | 27 | 27 |
| 0.6 | NaAc | 1% | 11 | 18 |
| 0.4 | AcOH | 0.1 M | 3 | 13 |

¹AFX = acetate-free xanthan
²OX ACID = oxalic acid
³AcOH, NaAc = Acetic acid and sodium acetate
⁴In this case, 1% NaCl was included with the acetic acid and sodium acetate
⁵The haloacetamide was 2-Br-2-CN—N,N-dimethylacetamide.

Preferably, the acetate-free xanthan gum will be the only suspending agent However, it may be used in combination with various inorganic salts with which it and the buffer are compatible.

We claim:

1. An antimicrobial composition comprising, in water, a haloacetamide, an acetate-free Xanthan gum, and a buffer of sodium acetate and acetic acid in an amount effective to maintain a pH in the range of 1-5.

2. A composition of claim 1 in the form of a suspension.

3. A composition of claim 1 wherein said haloacetamide is 2,2 dibromo 3-nitrilipropionamide.

4. A composition of claim 1 wherein said haloacetamide is 2-bromo-2-cyano-N,N-dimethylacetamide.

5. A composition of claim 1 wherein said acetate-free Xanthan gum contains no more than 1.2% acetic acid or acetate groups by weight.

6. A composition of claim 1 wherein said buffer comprises sodium acetate and acetic acid in a weight ratio of 1.5:1 to 2.5:1.

7. A suspension of at least 5% by weight of a haloacetamide in water including acetate-free Xanthan gum suspending agent, and a buffer comprising 1-2% sodium acetate and 0.5-1% acetic acid.

8. A suspension of claim 7 wherein said haloacetamide comprises 10% to 45% haloacetamide, and said buffer is present in an amount effective to maintain a pH in said suspension of 1-5.

9. A suspension of claim 7 wherein said sodium acetate and acetic acid are present in a molar ratio of 1.5:1 to 2.5:1.

10. A suspension of claim 7 wherein said haloacetamide is present in an amount from 5% by weight to 60% by weight.

11. A suspension of claim 7 wherein said haloacetamide is present in an amount from 10% to 45% by weight and said buffer is effective to maintain the pH at 3.8-4.2.

12. A suspension of claim 7 wherein said haloacetamide is present in an amount from 15% to 25% by weight.

13. A suspension of claim 7 wherein said acetate-free Xanthan gum is present in an amount from 0. 1% to 5% by weight.

14. A suspension of claim 7 wherein said acetate-free Xanthan gum is present in an amount from 0.5% to 4% by weight.

15. A stable antimicrobial composition comprising water, a haloacetamide in an effective antimicrobial amount, an acetate-free Xanthan gum in an amount effective to form a suspension of said haloacetamide in said water, sodium acetate, and acetic acid, said sodium acetate and acetic acid being present in a ratio and amount effective to inhibit the degradation of said haloacetamide by hydrolysis.

16. A composition of claim 15 wherein said haloacetamide is 2,2 dibromo 3-nitrilipropionamide.

17. A composition of claim 16 wherein said 2,2 dibromo 3-nitrilopropionamide is present in an amount from 5% to 60% by weight.

18. A composition of claim 17 wherein said sodium acetate and acetic acid are present in a molar ratio of 1.5:1 to 2.5.
